(19) United States
(12) Patent Application Publication
(10) Pub. No.: US 2014/0308720 A1
(43) Pub. Date: Oct. 16, 2014

(54) PROCESSES FOR PRODUCING LEVULINIC ACID FROM BIOMASS

(71) Applicant: API Intellectual Property Holdings, LLC, Atlanta, GA (US)

(72) Inventors: Theodora RETSINA, Atlanta, GA (US); Vesa PYLKKANEN, Atlanta, GA (US); Ryan O'CONNOR, Minnetrista, MN (US)

(73) Assignee: API Intellectual Property Holdings, LLC, Atlanta, GA (US)

(21) Appl. No.: 14/250,989

(22) Filed: Apr. 11, 2014

Related U.S. Application Data
(60) Provisional application No. 61/810,767, filed on Apr. 11, 2013.

(51) Int. Cl. C07C 51/36; C12P 7/40

(52) U.S. Cl. CPC C07C 51/36; C12P 7/40
USPC 435/136; 562/577

(57) ABSTRACT

This invention provides processes to convert biomass, including wood and agricultural residues, to levulinic acid and co-products. Some variations treat feedstock with steam and/or hot water to produce an extract liquor containing hemicellulosic oligomers, lignin, and cellulose-rich solids, wherein the hemicellulosic oligomers comprise C₅ hemicelluloses and C₆ hemicelluloses; separate the cellulose-rich solids from the extract liquor, to produce dewatered solids containing cellulose and lignin; dehydrate the hemicellulosic oligomers to convert the C₆ hemicelluloses directly to 5-hydroxymethylfurfural; and convert the 5-hydroxymethylfurfural to levulinic acid. Also, the cellulose may be dehydrated directly to 5-hydroxymethylfurfural, which may then be converted to additional levulinic acid. Various biorefinery embodiments are disclosed, in which C₅ and C₆ sugars are processed separately or in combination.

### PRIORITY DATA

This non-provisional patent application claims priority to U.S. Provisional Patent App. No. 61/810,767 for “PROCESSES AND APPARATUS FOR PRODUCING FURFURAL, LEVULINIC ACID, AND OTHER SUGAR-DERIVED PRODUCTS FROM BIOMASS,” filed Apr. 11, 2013, which is hereby incorporated by reference herein in its entirety.

### FIELD OF THE INVENTION

The present invention generally relates to processes for converting lignocellulosic biomass into various chemicals derived from sugars, including furfural and levulinic acid.

### BACKGROUND OF THE INVENTION

Cellulose and starch are polymers made of carbohydrate molecules, predominantly glucose, galactose, or other hexoses. When subjected to acid treatment, cellulose and starch hydrolyze into hexose monomers. On continued reaction, the hexose monomers further react to hydroxymethylfurfural, and other reaction intermediates, which then can further react to levulinic acid and formic acid. Levulinic acid can be produced by heating hexose, or any carbohydrate containing hexose, with a dilute mineral acid for an extended time.

Levulinic acid (C₅H₈O₃) is a short-chain fatty acid having a ketone carbonyl group and an acidic carboxyl group. It is a versatile platform chemical with numerous potential uses. For example, levulinic acid can be used to make resins, plasticizers, specialty chemicals, herbicides, fuels, and fuel additives.

The U.S. Department of Energy has identified levulinic acid as an important building-block chemical for biorefineries. The family of compounds that can be produced from levulinic acid is quite broad and addresses a number of large-volume chemical markets. Also, conversion of levulinic acid to methyltetrahydrofuran and various levulinate esters addresses fuel markets as gasoline and biodiesel additives, respectively. See Werpy, et al., “Top Value Added Chemicals From Biomass. Volume 1—Results of Screening for Potential Candidates From Sugars and Synthesis Gas”, U.S. Department of Energy, Washington, D.C., 2004, which is hereby incorporated by reference. According to the DOE report, the technical barriers for this building block include improvement of the process for levulinic acid production itself

Many materials such as glucose, sucrose, fructose, and biomass materials including wood, starch, cane sugar, grain sorghum, and agricultural wastes have been used to produce levulinic acid. Sugars are converted to levulinic acid essentially by a process of dehydration and cleavage of a mole of formic acid. Under acidic condition at elevated temperatures, carbohydrate decomposition can result in a variety of products, with levulinic acid and formic acid being the final soluble products from hexoses through an intermediate, 5-hydroxymethyl-2-furfural (5-HMF).

Likewise, pentose sugars can react to produce furfural. Under conditions of heat and acid, xylose and other five-carbon sugars undergo dehydration, losing three water molecules to become furfural (C₅H₄O₂). Furfural is an important renewable, non-petroleum based, chemical feedstock. Hydrogenation of furfural provides furfuryl alcohol, which is a useful chemical intermediate and which may be further hydrogenated to tetrahydrofurfuryl alcohol. Furfural is used to make other furan chemicals, such as furoic acid, via oxidation, and furan via decarbonylation.

Often furfural and levulinic acid are regarded as degradation products to be avoided, especially when biomass sugars are to be fermented. However, on-purpose production of furfural and/or levulinic acid, and/or precursors or derivatives thereof, can be of significant commercial interest from the sugar platform. Improved biorefinery processes, apparatus, and systems to produce furfural, levulinic acid, and related chemical intermediates are needed.

### SUMMARY OF THE INVENTION

Some variations provide a process for producing levulinic acid from cellulosic biomass, the process comprising:
(a) providing a feedstock comprising cellulosic biomass;
(b) providing an extraction solution comprising steam and/or hot water;
(c) treating the feedstock with the extraction solution under effective extraction conditions to produce an extract liquor containing hemicellulosic oligomers, lignin, and cellulose-rich solids, wherein the hemicellulosic oligomers comprise C₅ hemicelluloses and C₆ hemicelluloses;
(d) separating at least a portion of the cellulose-rich solids from the extract liquor, to produce dewatered solids containing cellulose and lignin;
(e) dehydrating the hemicellulosic oligomers under effective dehydration conditions to convert at least a portion of the C₆ hemicelluloses directly to 5-hydroxymethylfurfural;
(f) converting at least some of the 5-hydroxymethylfurfural to levulinic acid; and
(g) recovering the levulinic acid.

In some embodiments, the extraction solution further comprises an extraction additive, such as an acid or acid derivative. In some embodiments, step (e) utilizes a dehydration catalyst, which may be an acid catalyst, a base catalyst, a metal catalyst, or a metal oxide catalyst, for example. In certain embodiments, the extraction solution further comprises an acid, and the dehydration catalyst comprises that acid or a derivative thereof.

In some embodiments, the effective dehydration conditions convert at least a portion of the C₅ hemicelluloses to furfural, either directly or indirectly (i.e. by first generating hemicellulose monomers). In certain embodiments, the process further comprises converting at least a portion of the furfural to additional levulinic acid by a combination of hydration and hydrogenation. The hydrogenation may utilize hydrogen from syngas obtained from gasification of the biomass and/or the lignin, or other sources of hydrogen.

During step (e), the effective dehydration conditions may hydrolyze a portion of the hemicellulosic oligomers to hemicellulose monomers. The hemicellulose monomers may then be dehydrated into furfural and/or 5-hydroxymethylfurfural.

In some embodiments, the process further comprises dehydrating at least a portion of the cellulose, from step (d), directly to cellulose-derived 5-hydroxymethylfurfural; and then converting at least a portion of the cellulose-derived 5-hydroxymethylfurfural to levulinic acid.

In these or other embodiments, process further comprises hydrolyzing at least a portion of the cellulose, from step (d), to glucose; dehydrating at least a portion of the glucose to cellulose-derived 5-hydroxymethylfurfural; and then converting at least a portion of the cellulose-derived 5-hydroxymethylfurfural to levulinic acid by ring-opening hydration of the 5-hydroxymethylfurfural.

Other variations of the invention provide a process for producing levulinic acid from cellulosic biomass, the process comprising:
(a) providing a feedstock comprising cellulosic biomass;
(b) providing an extraction solution comprising steam and/or hot water;
(c) treating the feedstock with the extraction solution under effective extraction conditions to produce an extract liquor containing hemicellulosic oligomers, lignin, and cellulose-rich solids, wherein the hemicellulosic oligomers comprise C₅ hemicelluloses and C₆ hemicelluloses;
(d) separating at least a portion of the cellulose-rich solids from the extract liquor, to produce dewatered solids containing cellulose and lignin;
(e) dehydrating at least a portion of the cellulose directly to cellulose-derived 5-hydroxymethylfurfural;
(f) converting at least a portion of the cellulose-derived 5-hydroxymethylfurfural to levulinic acid; and
(g) recovering the levulinic acid.

In some embodiments, the extraction solution further comprises an extraction additive. In some embodiments, step (e) utilizes a dehydration catalyst. The dehydration catalyst may comprise an acid (contained in the extraction solution) or a derivative thereof.

In some embodiments, the process further comprises converting at least a portion of the C₆ hemicelluloses directly to 5-hydroxymethylfurfural. This 5-hydroxymethylfurfural may then be converted to additional levulinic acid by ring-opening hydration of the 5-hydroxymethylfurfural.

In certain embodiments, the process further comprises converting at least a portion of the C₅ hemicelluloses directly to furfural, or indirectly by first generating monomers and dehydrating the monomers to furfural. This furfural may be converted to additional levulinic acid by a combination of hydration and hydrogenation, wherein the hydrogenation optionally utilizes hydrogen from syngas obtained from gasification of the biomass and/or the lignin. In these or other embodiments, the process further comprises converting at least a portion of the hemicellulosic oligomers to hemicellulose monomers, and then fermenting the monomers to additional levulinic acid. Glucose from cellulose hydrolysis may also be fermented to additional levulinic acid, if desired.

### BRIEF DESCRIPTION OF THE FIGURES

FIG. 1 is a simplified block-flow diagram depicting the process of some embodiments of the present invention, in which levulinic acid is generated from both cellulose and hemicellulose.

FIG. 2 is a simplified block-flow diagram depicting the process of some embodiments of the present invention, in which levulinic acid is generated from hemicellulose while cellulose is recovered or further processed for other purposes.

FIG. 3 is a simplified block-flow diagram depicting the process of some embodiments of the present invention, in which levulinic acid is generated from cellulose while hemicellulose is recovered or further processed for other purposes.

### DISCLOSURE OF EMBODIMENTS OF THE INVENTION

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”

For purposes of an enabling technical disclosure, various explanations, hypotheses, theories, speculations, assumptions, and so on are disclosed. The present invention does not rely on any of these being in fact true. None of the explanations, hypotheses, theories, speculations, or assumptions in this detailed description shall be construed to limit the scope of the invention in any way.

Certain exemplary embodiments of the invention will now be described. These embodiments are not intended to limit the scope of the invention as claimed. The order of steps may be varied, some steps may be omitted, and/or other steps may be added. Any reference herein to first step, second step, etc. is for illustration purposes only.

Some variations of the invention are premised on the realization that (i) chemical conversion of sugars can be useful for certain desired products and (ii) integrated processes for efficient production of biomass sugars can be utilized to directly or indirectly convert the biomass sugars into a wide variety of chemicals, in one or multiple steps.

Some variations provide a process for producing levulinic acid from cellulosic biomass, the process comprising:
(a) providing a feedstock comprising cellulosic biomass;
(b) providing an extraction solution comprising steam and/or hot water;
(c) treating the feedstock with the extraction solution under effective extraction conditions to produce an extract liquor containing hemicellulosic oligomers, lignin, and cellulose-rich solids, wherein the hemicellulosic oligomers comprise C₅ hemicelluloses and C₆ hemicelluloses;
(d) separating at least a portion of the cellulose-rich solids from the extract liquor, to produce dewatered solids containing cellulose and lignin;
(e) dehydrating the hemicellulosic oligomers under effective dehydration conditions to convert at least a portion of the C₆ hemicelluloses directly to 5-hydroxymethylfurfural;
(f) converting at least some of the 5-hydroxymethylfurfural to levulinic acid; and
(g) recovering the levulinic acid.

In some embodiments, the extraction solution further comprises an extraction additive, such as an acid or acid derivative. In some embodiments, step (e) utilizes a dehydration catalyst, which may be an acid catalyst, a base catalyst, a metal catalyst, or a metal oxide catalyst, for example. In certain embodiments, the extraction solution further comprises an acid, and the dehydration catalyst comprises that acid or a derivative thereof.

In some embodiments, the effective dehydration conditions convert at least a portion of the C₅ hemicelluloses to furfural, either directly or indirectly (i.e. by first generating hemicellulose monomers). In certain embodiments, the process further comprises converting at least a portion of the furfural to additional levulinic acid by a combination of hydration and hydrogenation. The hydrogenation may utilize hydrogen from syngas obtained from gasification of the biomass and/or the lignin, or other sources of hydrogen.

During step (e), the effective dehydration conditions may hydrolyze a portion of the hemicellulosic oligomers to hemicellulose monomers. The hemicellulose monomers may then be dehydrated into furfural and/or 5-hydroxymethylfurfural.

In some embodiments, the process further comprises dehydrating at least a portion of the cellulose, from step (d), directly to cellulose-derived 5-hydroxymethylfurfural; and then converting at least a portion of the cellulose-derived 5-hydroxymethylfurfural to levulinic acid.

In these or other embodiments, process further comprises hydrolyzing at least a portion of the cellulose, from step (d), to glucose; dehydrating at least a portion of the glucose to cellulose-derived 5-hydroxymethylfurfural; and then converting at least a portion of the cellulose-derived 5-hydroxymethylfurfural to levulinic acid by ring-opening hydration of the 5-hydroxymethylfurfural.

Other variations of the invention provide a process for producing levulinic acid from cellulosic biomass, the process comprising:
(a) providing a feedstock comprising cellulosic biomass;
(b) providing an extraction solution comprising steam and/or hot water;
(c) treating the feedstock with the extraction solution under effective extraction conditions to produce an extract liquor containing hemicellulosic oligomers, lignin, and cellulose-rich solids, wherein the hemicellulosic oligomers comprise C₅ hemicelluloses and C₆ hemicelluloses;
(d) separating at least a portion of the cellulose-rich solids from the extract liquor, to produce dewatered solids containing cellulose and lignin;
(e) dehydrating at least a portion of the cellulose directly to cellulose-derived 5-hydroxymethylfurfural;
(f) converting at least a portion of the cellulose-derived 5-hydroxymethylfurfural to levulinic acid; and
(g) recovering the levulinic acid.

In some embodiments, the extraction solution further comprises an extraction additive. In some embodiments, step (e) utilizes a dehydration catalyst. The dehydration catalyst may comprise an acid (contained in the extraction solution) or a derivative thereof.

In some embodiments, the process further comprises converting at least a portion of the C₆ hemicelluloses directly to 5-hydroxymethylfurfural. This 5-hydroxymethylfurfural may then be converted to additional levulinic acid by ring-opening hydration of the 5-hydroxymethylfurfural.

In certain embodiments, the process further comprises converting at least a portion of the C₅ hemicelluloses directly to furfural, or indirectly by first generating monomers and dehydrating the monomers to furfural. This furfural may be converted to additional levulinic acid by a combination of hydration and hydrogenation, wherein the hydrogenation optionally utilizes hydrogen from syngas obtained from gasification of the biomass and/or the lignin. In these or other embodiments, the process further comprises converting at least a portion of the hemicellulosic oligomers to hemicellulose monomers, and then fermenting the monomers to additional levulinic acid. Glucose from cellulose hydrolysis may also be fermented to additional levulinic acid, if desired.

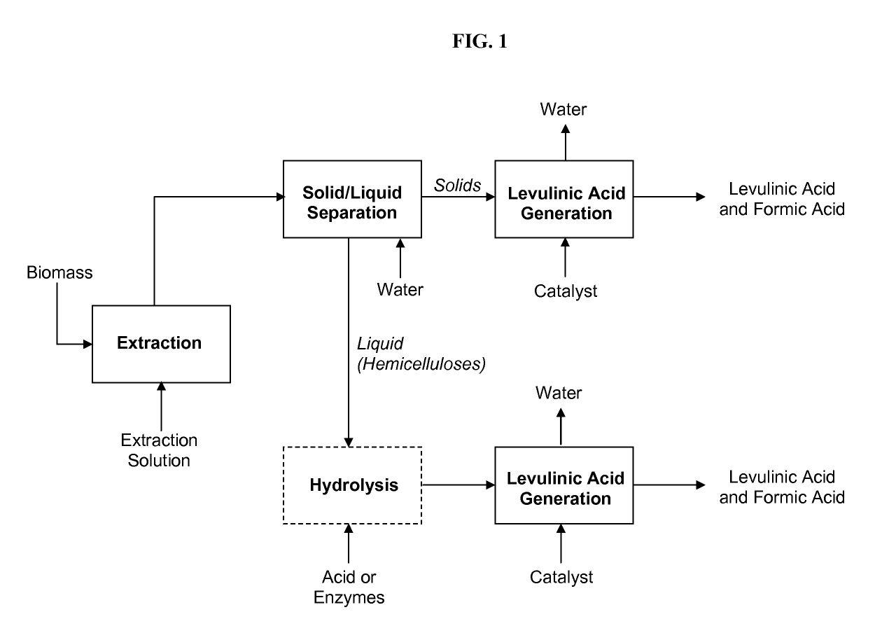

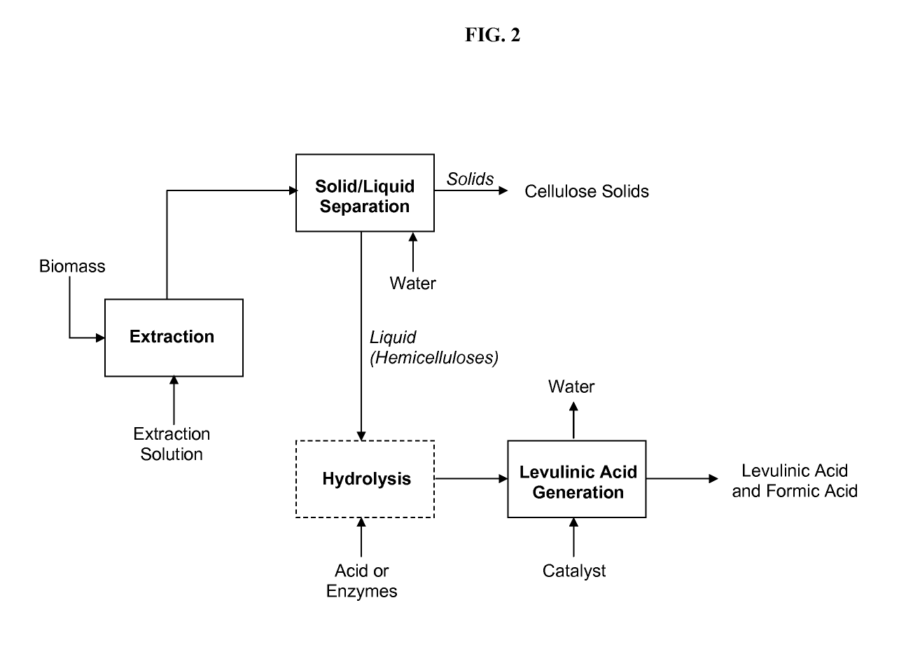

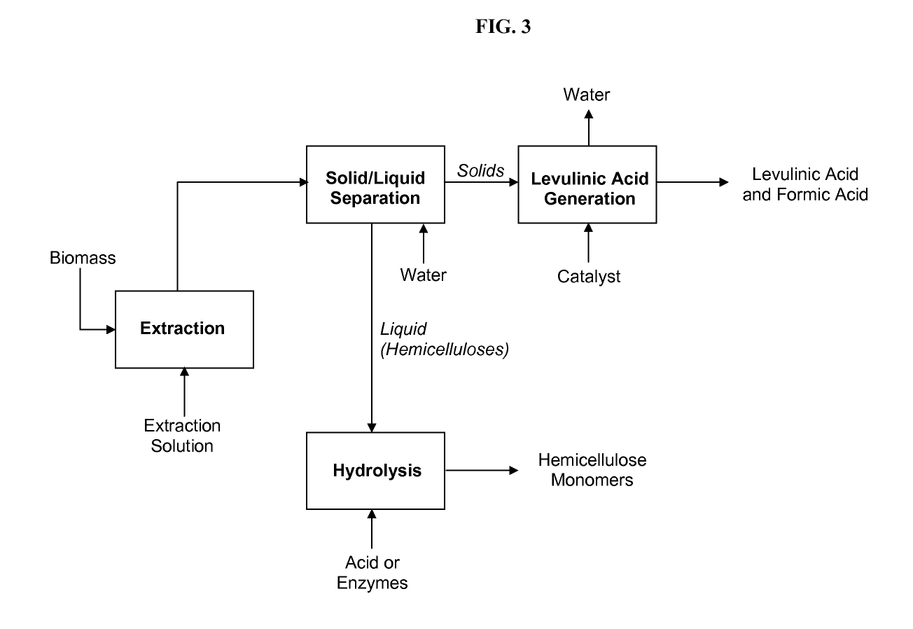

FIG. 1 is a simplified block-flow diagram depicting the process of some embodiments of the present invention, in which levulinic acid is generated from both cellulose and hemicellulose. FIG. 2 is a simplified block-flow diagram depicting the process of some embodiments of the present invention, in which levulinic acid is generated from hemicellulose while cellulose is recovered or further processed for other purposes (such as combustion, pulping, nanocellulose production, etc.). FIG. 3 is a simplified block-flow diagram depicting the process of some embodiments of the present invention, in which levulinic acid is generated from cellulose while hemicellulose is recovered or further processed for other purposes (such as fermentation, combustion, etc.). In any of these configurations, 5-hydromethylfurfural and/or furfural may be produced and recovered instead of, or in addition to, levulinic acid.

In some variations, Green Power+® technology, commonly assigned with the assignee of this patent application, may be employed or modified to adjust the process toward furfural, 5-hydromethylfurfural, and/or levulinic acid. Some embodiments employ reaction conditions and operation sequences described in U.S. Pat. No. 8,211,680, issued Jul. 3, 2012; and/or U.S. Patent Application Ser. Nos. 13/471,662; 13/026,273; 13/026,280; 13/500,917; 61/536,477; 61/612,451; 61/612,453; 61/624,880; 61/638,730; 61/641,435; 61/679,793; 61/696,360; 61/709,960. Each of these commonly owned patents and patent applications is hereby incorporated by reference herein in its entirety.

Generally speaking, process conditions that may be adjusted to promote furfural, 5-hydromethylfurfural, and/or levulinic acid include, in one or more reaction steps, temperature, pH or acid concentration, reaction time, catalysts or other additives (e.g. FeSO₄), reactor flow patterns, and control of engagement between liquid and vapor phases. Conditions may be optimized specifically for furfural, or specifically for 5-hydromethylfurfural, or specifically for levulinic acid, or for any combination thereof.

“Biomass,” for purposes of this disclosure, shall be construed as any biogenic feedstock or mixture of a biogenic and non-biogenic feedstock. Elementally, biomass includes at least carbon, hydrogen, and oxygen. The methods and apparatus of the invention can accommodate a wide range of feedstocks of various types, sizes, and moisture contents.

Biomass includes, for example, plant and plant-derived material, vegetation, agricultural waste, forestry waste, wood waste, paper waste, animal-derived waste, poultry-derived waste, and municipal solid waste. In various embodiments of the invention utilizing biomass, the biomass feedstock may include one or more materials selected from: softwood chips, hardwood chips, timber harvesting residues, tree branches, tree stumps, knots, leaves, bark, sawdust, off-spec paper pulp, cellulose, corn, corn stover, wheat straw, rice straw, sugarcane, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, carbohydrates, plastic, and cloth.

Selection of a particular feedstock or feedstocks is not regarded as technically critical, but is carried out in a manner that tends to favor an economical process. Typically, regardless of the feedstocks chosen, there can be (in some embodiments) screening to remove undesirable materials. The feedstock can optionally be dried prior to processing.

The feedstock employed may be provided or processed into a wide variety of particle sizes or shapes. For example, the feed material may be a fine powder, or a mixture of fine and coarse particles. The feed material may be in the form of large pieces of material, such as wood chips or other forms of wood (e.g., round, cylindrical, square, etc.). In some embodiments, the feed material comprises pellets or other agglomerated forms of particles that have been pressed together or otherwise bound, such as with a binder.

In some embodiments, biomass may be first extracted with steam or liquid hot water, to remove at least a portion of the hemicelluloses that are present in the starting material. The liquid hot water may include process condensate from one or more downstream steps.

The hemicelluloses that were initially extracted may then be processed to produce furfural and 5-hydroxymethylfurfural (HMF), in one or more steps. Some furfural and HMF may be produced during the initial extraction itself, under suitable conditions. In some embodiments, the hemicellulose-containing liquor is fed to a unit for production of furfural directly from C₅ monomers and oligomers and HMF directly from C₆ monomers and oligomers. That is, without being limited to any hypothesis, it is believed that furfural and HMF may be produced directly from an oligomeric sugar molecule, rather than from a monomeric sugar.

On the other hand, in some embodiments, it may be preferable to first produce a relatively high fraction of monomers prior to producing furfural and HMF. This configuration may offer kinetic benefits to avoid competing reaction pathways, in parallel or in series. Namely, when starting with primarily monomeric pentoses and hexoses, the conditions may be tuned to optimize furfural and HMF. When starting with a distribution of chain lengths, reactions to hydrolyze the oligomers into monomers may compete kinetically with dehydration reactions that form furfural and HMF. In order to reach high conversions of sugar oligomers, degradation, polymerization, or other reactions of furfural and HMF may take place, reducing the selectivity and yield to the desired products.

Thus in some embodiments, the hemicelluloses are first subject to a step to further hydrolyze the oligomers into monomers. This step may be performed with acids or enzymes. Depending on the feedstock, the hydrolyzed hemicelluloses will contain various quantities of C₅ sugars (e.g., xylose) and C₆ sugars (e.g., glucose).

In some embodiments, a reaction step is optimized to produce furfural. In some embodiments, a reaction step is optimized instead to produce HMF. In certain embodiments, a reaction step is configured to produce both furfural and HMF, which may be then separated or may be further processed together.

When it is desired to produce levulinic acid, the liquid may be further processed to convert at least some of the HMF into levulinic acid, with or without intermediate separation of furfural. In some embodiments, a reaction step is optimized to produce furfural, which is then recovered, followed by production of levulinic acid, which is separately recovered. In some embodiments, a single step is configured to produce both furfural and levulinic acid, which may be recovered together in a single liquid or may be separated from each other and then recovered. Conversion of HMF to levulinic acid also produces formic acid, which may be separately recovered, recycled, or purged.

In some embodiments, the furfural is further reacted, in the same reactor or in a downstream unit, to one or more acids such as succinic acid, maleic acid, fumaric acid, or humic acid. In some embodiments, conditions are selected to maximize conversion of furfural to succinic acid.

In various embodiments, the process is configured to produce, in crude or purified form, one or more products selected from the group consisting of levulinic acid, furfural, 5-hydroxymethylfurfural, formic acid, succinic acid, maleic acid, fumaric acid, and acetic acid. Mixtures of any of the foregoing are possible.

Any of the above-mentioned acids may be recycled in the process, such as to enhance the initial extraction of hemicelluloses or to enhance secondary hydrolysis of hemicellulose oligomers to monomers. Thus in some embodiments, acetic acid, formic acid, or other acids may be recovered and recycled.

Reaction conditions for producing furfural, HMF, and levulinic acid may vary widely (see, for example, U.S. Pat. Nos. 3,701,789 and 4,897,497 for some conditions that may be used). Temperatures may vary, for example, from about 120° C. to about 275° C., such as about 200° C. to about 230° C. Reaction times may vary from less than 1 minute to more than 1 hour, including about 1, 2, 3, 5, 10, 15, 20, 30, 45, and 60 minutes. The quantity of acid may vary widely, depending on other conditions, such as from about 0.1% to about 10% by weight, e.g. about 0.5%, about 1%, or about 2% acid. The acid may include sulfuric acid, sulfurous acid, sulfur dioxide, formic acid, levulinic acid, succinic acid, maleic acid, fumaric acid, acetic acid, or lignosulfonic acid, for example.

The residence times of the reactors may vary. There is an interplay of time and temperature, so that for a desired amount of hydrolysis or dehydration, higher temperatures may allow for lower reaction times, and vice versa. The residence time in a continuous reactor is the volume divided by the volumetric flow rate. The residence time in a batch reactor is the batch reaction time, following heating to reaction temperature.

The mode of operation for the reactor, and overall system, may be continuous, semi-continuous, batch, or any combination or variation of these. In some embodiments, the reactor is a continuous, countercurrent reactor in which solids and liquid flow substantially in opposite directions. The reactor may also be operated in batch but with simulated countercurrent flow.

When multiple stages are utilized, such as a first stage to produce or optimize furfural and HMF followed by a second stage to produce or optimize levulinic acid, the conditions of the second stage may be the same as in the first stage, or may be more or less severe. If furfural is removed, at least in part, a quantity of acid may also be removed (e.g. by evaporation) in which case it may be necessary to introduce an additional amount of acid to the second stage.

The remaining solids, rich in cellulose and lignin, may be used in a number of ways including for power production, pellet production, or pulp production (including market pulp, dissolving pulp, and fluff pulp), for example. In some embodiments, the solids are subjected to one or more steps to remove at least some of the lignin prior to pulping or cellulose hydrolysis. Lignin removal may be accomplished using chemical bleaching or enzymatic lignin oxidation, for example.

Certain exemplary embodiments will now be described in detail, without limitation of this disclosure.

In some embodiments, such as the process depicted in FIG. 1, the process starts as biomass is received or reduced to approximately ¼″ thickness. In a first step of the process, the biomass chips are fed to a pressurized extraction vessel operating continuously or in batch mode. The chips may be steamed or water-washed to remove dirt and entrained air. The chips are immersed with aqueous liquor or saturated vapor and heated to a temperature between about 100° C. to about 250° C., for example 150° C., 160° C., 170° C., 180° C., 190° C., 200° C., or 210° C. Preferably, the chips are heated to about 180° C. to 210° C. The pressure in the pressurized vessel may be adjusted to maintain the aqueous liquor as a liquid, a vapor, or a combination thereof. Exemplary pressures are about 1 atm to about 30 atm, such as about 3 atm, 5 atm, 10 atm, or 15 atm.

The aqueous liquor may contain acidifying compounds, such as (but not limited to) sulfuric acid, sulfurous acid, sulfur dioxide, acetic acid, formic acid, or oxalic acid, or combinations thereof. The dilute acid concentration can range from 0.01% to 10% as necessary to improve solubility of particular minerals, such as potassium, sodium, or silica. Preferably, the acid concentration is selected from about 0.01% to 4%, such as 0.1%, 0.5%, 1%, 1.5%, 2%, 2.5%, 3%, or 3.5%.

A second step may include depressurization of the extracted chips. The vapor can be used for heating the incoming woodchips or cooking liquor, directly or indirectly. The volatilized organic acids (e.g., acetic acid or formic acid), which are generated or included in the cooking step, may be recycled back to the cooking via process condensate or other means.

A third step may include washing the extracted chips. The washing may be accomplished with water, recycled condensates, recycled permeate, or combination thereof. A liquid biomass extract is produced. A countercurrent configuration may be used to maximize the biomass extract concentration. Washing typically removes most of the dissolved material, including hemicelluloses and minerals. The final consistency of the washing may be increased to 30% or more, preferably to 50% or more, using a mechanical pressing device. In some embodiments, washing is performed in the same unit as steam or liquid hot-water extraction. An additional washing unit may still be used, if desired.

A fourth step may include drying of the extracted material to a desired final moisture. The heat necessary for drying may be derived from combusting part of the starting biomass. Alternatively, or additionally, the heat for drying may be provided by other means, such as a natural gas boiler or other auxiliary fossil fuel, or from a waste heat source. Optionally, drying of the extracted material may be accomplished by pyrolysis, torrefaction (mild pyrolysis), or gasification of the extracted material.

A fifth step may include preparing the biomass for combustion. This step may include grinding, milling, fluidizing, and/or pelletizing the extracted biomass. The biomass may be fed to a boiler in the form of fine powder, loose fiber, pellets, briquettes, or any other suitable form. In some embodiments, pellets of extracted biomass (“biomass pellets”) are preferred.

A sixth step may be combustion of the biomass, which in some embodiments is in the form of biomass pellets. The biomass pellets are fed to boiler and combusted, preferably with excess air, using well-known combustion apparatus. Boiler bottom may be fixed, moving, or fluidized for the best efficiency. The flue gas is cooled and fly ash is collected into gravity collectors. In some embodiments, the extracted biomass is sufficiently low in ash such that when the extracted biomass is combusted, particulate matter emissions are very low. In certain embodiments, the particulate matter emissions are so low as to avoid the need for any additional cleaning device, and associated control system, in order to meet current emission regulations.

A seventh step may include treatment of the biomass extract to form a hydrolysate comprising hemicellulose sugars. In some embodiments, the biomass extract is hydrolyzed using dilute acidic conditions at temperatures between about 100° C. and 190° C., for example about 120° C., 130° C., 140° C., 150° C., 160° C., or 170° C., and preferably from 120° C. to 150° C.

The acid may be selected from sulfuric acid, sulfurous acid, or sulfur dioxide. Alternatively, or additionally, the acid may include formic acid, acetic acid, or oxalic acid from the cooking liquor or recycled from previous hydrolysis. Alternatively, hemicellulase enzymes may used instead of acid hydrolysis. The lignin from this step may be separated and recovered, or recycled to increase the heating value of the pellets, or sent directly to the boiler.

An eighth step may include evaporation of hydrolyzate to remove some or most of the volatile acids. The evaporation may include flashing or stripping to remove sulfur dioxide, if present, prior to removal of volatile acids. The evaporation step is preferably performed below the acetic acid dissociation pH of 4.8, and most preferably a pH selected from about 1 to about 2.5. The dissolved solids may be concentrated, such as to about 10% to about 40%.

In some embodiments, additional evaporation steps may be employed. These additional evaporation steps may be conducted at different conditions (e.g., temperature, pressure, and pH) relative to the first evaporation step. Any evaporation steps employed herein may utilize mechanical vapor recompression evaporation.

In some embodiments, some or all of the organic acids evaporated may be recycled, as vapor or condensate, to the first step (cooking step) and/or third step (washing step) to remove assist in the removal of minerals from the biomass. This recycle of organic acids, such as acetic acid, may be optimized along with process conditions that may vary depending on the amount recycled, to improve the cooking and/or washing effectiveness.

The acetic acid that is vaporized may be converted to an acetate salt and recovered. For example, potassium acetate may be produced as a co-product. In some embodiments, ethyl acetate is produced as a co-product.

In certain embodiments, the process further comprises combining, at a pH of about 4.8 to 10 or higher, a portion of the vaporized acetic acid with an alkali oxide, alkali hydroxide, alkali carbonate, and/or alkali bicarbonate, wherein the alkali is selected from the group consisting of potassium, sodium, magnesium, calcium, and combinations thereof, to convert the portion of the vaporized acetic acid to an alkaline acetate. The alkaline acetate may be recovered. If desired, purified acetic acid may be generated from the alkaline acetate.

Optionally, the process may include co-combusting the recovered lignin with the low-ash biomass, to produce power. The recovered lignin may be combined with the low-ash biomass prior to combustion, or they may be co-fired as separate streams. When recovered lignin is combined with the low-ash biomass for making pellets, the lignin can act as a pellet binder.

In some embodiments, the hemicellulose sugars are converted to furfural, HMF, and/or levulinic acid in various quantities. In some embodiments, a portion of the hemicellulose sugars are separately fermented to ethanol, 1-butanol, isobutanol, acetic acid, lactic acid, succinic acid, or any other fermentation products.

A purified product may be produced by distillation, which will also generate a distillation bottoms stream containing residual solids. A bottoms evaporation stage may be used, to produce residual solids. Residual solids (such as distillation bottoms) may be recovered, or burned in solid or slurry form, or recycled to be combined into the biomass pellets.

Part or all of the residual solids may be co-combusted with the low-ash biomass, if desired. Alternatively, or additionally, the process may include recovering the residual solids as a co-product in solid, liquid, or slurry form. The co-product may be used as a fertilizer or fertilizer component, since it will typically be rich in potassium, nitrogen, and/or phosphorous.

In certain embodiments, the process further comprises combining, at a pH of about 4.8 to 10 or higher, a portion of the vaporized acetic acid with an alkali oxide, alkali hydroxide, alkali carbonate, and/or alkali bicarbonate, wherein the alkali is selected from the group consisting of potassium, sodium, magnesium, calcium, and combinations thereof, to convert the portion of the vaporized acetic acid to an alkaline acetate. The alkaline acetate may be recovered. If desired, purified acetic acid may be generated from the alkaline acetate.

In other embodiments, following extraction the solids (rich in cellulose) are not combusted but rather are hydrolyzed to glucose using enzyme or acid hydrolysis. The glucose may be fermented to a fermentation product such as ethanol, 1-butanol, isobutanol, acetic acid, lactic acid, succinic acid, or any other fermentation products.

In some embodiments, the glucose from solids hydrolysis is converted to levulinic acid, via HMF, using the principles disclosed herein. In some embodiments, the extracted material is fed to a unit in which HMF and then levulinic acid are directly produced from the cellulose-rich solids, without intermediate production of glucose (although glucose may be a reactive intermediate in situ).

In some embodiments, the extracted hemicelluloses are processed to maximize furfural production while the cellulose-rich solids are separately processed to maximize levulinic acid production.

In some embodiments, the cellulose-rich solids are processed to produce HMF, levulinic acid, or both of these, while the hemicellulose sugars are fermented (and not processed to intentionally produce furfural).

In some embodiments, hemicelluloses which contain C₅ and C₆ fractions are subjected to an intermediate separation. Such a separation may be accomplished by chromatographic separation, membranes, or other means.

Then the C₅-enriched fraction may be optimized for furfural production while the C₆-enriched fraction is optimized for HMF and/or levulinic acid production. Or the C₅-enriched fraction may be optimized for furfural production while the C₆-enriched fraction is optimized for hydrolysis to C₆ sugars for fermentation. Or the C₅-enriched fraction may be optimized for hydrolysis to C₅ sugars for fermentation while the C₆-enriched fraction is optimized for HMF and/or levulinic acid production. Following separation of C₅ and C₆ hemicellulose fractions, the C₆-enriched stream may be combined with a separate C₆ stream derived from the cellulose-rich solids, if desired.

In some embodiments in which levulinic acid is the target product, additional processing steps are included to convert furfural into levulinic acid. Although both furfural and levulinic acid are C₅ molecules, furfural has four fewer hydrogen atoms and one fewer oxygen atom compared to levulinic acid. Thus a combination of hydration and hydrogenation may convert furfural to levulinic acid. In certain embodiments, the hydrogen may be provided from syngas obtained from gasification of lignin that is derived from the initial biomass. In certain embodiments, hydrogen is obtained from syngas produced from cellulose-rich solids processed in an integrated gasification combined cycle plant that produces syngas primarily for power production.

Various separation schemes may be implemented to recover the furfural, HMF, and/or levulinic acid. In some embodiments, a distillation column or steam stripper is used. Separation techniques can include or use distillation columns, flash vessels, centrifuges, cyclones, membranes, filters, packed beds, capillary columns, and so on. Separation can be principally based, for example, on distillation, absorption, adsorption, or diffusion, and can utilize differences in vapor pressure, activity, molecular weight, density, viscosity, polarity, chemical functionality, affinity to a stationary phase, and any combinations thereof. In certain embodiments, vacuum distillation is employed.

The throughput, or process capacity, may vary widely from small laboratory-scale units to full commercial-scale biorefineries, including any pilot, demonstration, or semi-commercial scale. In various embodiments, the process capacity is at least about 1 lb/day, 10 lb/day, 100 lb/day, 1 ton/day, 10 tons/day, 100 tons/day, 500 tons/day, 1000 tons/day, 2000 tons/day, or higher.

The present invention preferably employs heat and mass integration within the biorefinery, and possibly with a co-located operation at the site. In some embodiments, a portion of any material produced may be recycled to the front end of the process, or to another upstream location. Solid, liquid, and gas streams produced or existing within the process can be independently recycled, passed to subsequent steps, or removed/purged from the process at any point.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure, it is the intent that this disclosure will cover those variations as well.

What is claimed is:

1. A process for producing levulinic acid from cellulosic biomass, said process comprising:
(a) providing a feedstock comprising cellulosic biomass;
(b) providing an extraction solution comprising steam and/or hot water;
(c) treating said feedstock with said extraction solution under effective extraction conditions to produce an extract liquor containing hemicellulosic oligomers, lignin, and cellulose-rich solids, wherein said hemicellulosic oligomers comprise C₅ hemicelluloses and C₆ hemicelluloses;
(d) separating at least a portion of said cellulose-rich solids from said extract liquor, to produce dewatered solids containing cellulose and lignin;
(e) dehydrating said hemicellulosic oligomers under effective dehydration conditions to convert at least a portion of said C₆ hemicelluloses directly to 5-hydroxymethylfurfural;
(f) converting at least some of said 5-hydroxymethylfurfural to levulinic acid; and
(g) recovering said levulinic acid.

2. The process of claim 1, wherein said extraction solution further comprises an extraction additive.

3. The process of claim 2, wherein said extraction additive is an acid.

4. The process of claim 1, wherein step (e) utilizes a dehydration catalyst.

5. The process of claim 4, wherein said extraction solution further comprises an acid, and wherein said dehydration catalyst comprises said acid or a derivative thereof.

6. The process of claim 1, wherein said effective dehydration conditions convert at least a portion of said C₅ hemicelluloses to furfural.

7. The process of claim 6, said process further comprising converting at least a portion of said furfural to additional levulinic acid by a combination of hydration and hydrogenation.

8. The process of claim 7, wherein said hydrogenation utilizes hydrogen from syngas obtained from gasification of said biomass and/or said lignin.

9. The process of claim 1, wherein during step (e), said effective dehydration conditions also hydrolyze a portion of said hemicellulosic oligomers to hemicellulose monomers, and wherein said hemicellulose monomers are then dehydrated into furfural and/or 5-hydroxymethylfurfural.

10. The process of claim 1, said process further comprising dehydrating at least a portion of said cellulose, from step (d), directly to cellulose-derived 5-hydroxymethylfurfural; and then converting at least a portion of said cellulose-derived 5-hydroxymethylfurfural to levulinic acid.

11. The process of claim 1, said process further comprising hydrolyzing at least a portion of said cellulose, from step (d), to glucose; dehydrating at least a portion of said glucose to cellulose-derived 5-hydroxymethylfurfural; and then converting at least a portion of said cellulose-derived 5-hydroxymethylfurfural to levulinic acid.

12. A process for producing levulinic acid from cellulosic biomass, said process comprising:
(a) providing a feedstock comprising cellulosic biomass;
(b) providing an extraction solution comprising steam and/or hot water;
(c) treating said feedstock with said extraction solution under effective extraction conditions to produce an extract liquor containing hemicellulosic oligomers, lignin, and cellulose-rich solids, wherein said hemicellulosic oligomers comprise C₅ hemicelluloses and C₆ hemicelluloses;
(d) separating at least a portion of said cellulose-rich solids from said extract liquor, to produce dewatered solids containing cellulose and lignin;
(e) dehydrating at least a portion of said cellulose directly to cellulose-derived 5-hydroxymethylfurfural;
(f) converting at least a portion of said cellulose-derived 5-hydroxymethylfurfural to levulinic acid; and
(g) recovering said levulinic acid.

13. The process of claim 12, wherein said extraction solution further comprises an extraction additive.

14. The process of claim 12, wherein step (e) utilizes a dehydration catalyst.

15. The process of claim 14, wherein said extraction solution further comprises an acid, and wherein said dehydration catalyst comprises said acid or a derivative thereof.

16. The process of claim 12, said process further comprising converting at least a portion of said C₆ hemicelluloses directly to 5-hydroxymethylfurfural.

17. The process of claim 16, said process further comprising converting at least a portion of said 5-hydroxymethylfurfural to additional levulinic acid.

18. The process of claim 12, said process further comprising converting at least a portion of said C₅ hemicelluloses directly to furfural.

19. The process of claim 18, said process further comprising converting at least a portion of said furfural to additional levulinic acid by a combination of hydration and hydrogenation, wherein said hydrogenation optionally utilizes hydrogen from syngas obtained from gasification of said biomass and/or said lignin.

20. The process of claim 12, said process further comprising converting at least a portion of said hemicellulosic oligomers to hemicellulose monomers, and then fermenting said monomers to additional levulinic acid.
